(19) United States
(12) Patent Application Publication
(10) Pub. No.: US 2023/0380850 A1
(43) Pub. Date: Nov. 30, 2023

(54) FUNNEL ASPIRATION CATHETER

(71) Applicant: Neuravi Limited, Galway (IE)

(72) Inventors: David VALE, Barna (IE); Kirk JOHNSON, Weston, FL (US)

(73) Assignee: Neuravi Limited, Galway (IE)

(21) Appl. No.: 17/824,992

(22) Filed: May 26, 2022

(51) Int. Cl. A61B 17/221; A61B 17/22

(52) U.S. Cl. CPC A61B 17/221; A61B 17/22; A61B 2017/22079

(57) ABSTRACT

Example systems and methods of treatment are presented herein which generally involve positioning a funnel aspiration catheter within a blood vessel such that a funnel mouth of the catheter is proximate an obstruction, aspirating in a proximal direction until the obstruction becomes lodged in the funnel mouth, and continuing to aspirate while simultaneously either torquing a guide wire, or injecting a solution through an injection catheter, where either the guide wire or injection catheter is positioned inside a lumen of the catheter. The use of either the guide wire or injection catheter, along with simultaneous aspiration, can help to macerate the obstruction while aspirating fragments of the obstruction in the proximal direction through the catheter.

### FIELD

The present invention generally relates to devices and methods for removing acute blockages from blood vessels during intravascular medical treatments. More specifically, the present disclosure relates to clot retrieval devices including a funnel aspiration catheter.

### BACKGROUND

Clot retrieval aspiration catheters and devices are used in mechanical thrombectomy for endovascular intervention, often in cases where patients are suffering from conditions such as acute ischemic stroke (AIS), myocardial infarction (MI), and pulmonary embolism (PE). Accessing the neurovascular bed in particular is challenging with conventional technology, as the target vessels are small in diameter, remote relative to the site of insertion, and highly tortuous. Traditional devices are often either too large in profile, lack the deliverability and flexibility needed to navigate particularly tortuous vessels, or are ineffective at removing a clot when delivered to the target site.

Conventional clot retrieval catheters suffer from a number of drawbacks. First, the diameters of catheters themselves must be small enough to avoid causing significant discomfort to the patient. The retrieval catheter must also be sufficiently flexible to navigate the vasculature and endure high strains, while also having the axial stiffness to offer smooth advancement along the route. Once at the target site, typical objects to be retrieved from the body are substantially larger in size than the catheter tip, making it more difficult to retrieve objects into the tip. For example, firm, fibrin-rich clots can often be difficult to extract as they can become lodged in the tip of traditional fixed-mouth catheters. Additionally, this lodging can cause softer portions to shear away from the firmer regions of the clot.

Small diameters and fixed tip sizes are also less efficient at directing the aspiration necessary to remove blood and thrombus material during the procedure. The suction must be strong enough such that any fragmentation that may occur as a result of aspiration or the use of a mechanical thrombectomy device can be held stationary so that fragments cannot migrate and occlude distal vessels. However, when aspirating with a fixed-mouth catheter, a significant portion of the aspiration flow ends up coming from vessel fluid proximal to the tip of the catheter, where there is no clot, because the diameter of the funnel catheter is smaller than that of the vessel. This significantly reduces aspiration efficiency, lowering the success rate of clot removal.

Additionally, conventional clot retrieval catheters are not always successful in fully ingesting a clot. In such cases, the clot can become “corked” or stuck in the tip of the catheter, requiring a physician to have to remove the catheter from the target site in order to retrieve the clot from the patient. During removal of the catheter, the portion of the clot remaining outside of the catheter tip can break off, becoming “uncorked” during retraction, and can go back downstream re-blocking the blood vessel. Re-gaining access to the treatment site can then be challenging.

Any catheter design attempting to overcome these challenges would need to be able to fully ingest a clot at a treatment site and aspirate the clot through the catheter while ensuring the clot, or pieces of the clot, would not become “uncorked” from the catheter tip.

The present design is aimed at providing an improved retrieval catheter which addresses the above-stated deficiencies.

### SUMMARY

Example systems and methods of treatment are presented herein which generally involve positioning a funnel aspiration catheter within a blood vessel such that a funnel mouth of the catheter is proximate an obstruction (e.g., a clot), aspirating in a proximal direction until the obstruction becomes lodged in the funnel mouth, and continuing to aspirate while simultaneously either torquing a guide wire, or injecting a solution through an injection catheter, where either the guide wire or injection catheter is positioned inside a lumen of the catheter. The use of either the guide wire or injection catheter can help to macerate the obstruction while aspirating fragments of the obstruction in the proximal direction through the catheter.

An example system configured to retrieve an obstruction from a blood vessel can include an aspiration catheter and a guide wire. The aspiration catheter can include an elongate body including a proximal end, a distal end, and a lumen sized to receive at least a first portion of the obstruction therein. The aspiration catheter can also include a funnel continuous with the distal end of the elongate body and including a funnel mouth sized to receive at least a second portion of the obstruction therein. The guide wire can be sized to slide through the lumen and can include one or more auger fins disposed at a distal end of the guide wire. The guide wire can be configured to be torqued such that the auger fins macerate the obstruction into fragments as one or more of the fragments of the obstruction are aspirated in a proximal direction through the aspiration catheter.

The elongate body of the aspiration catheter can have a single continuous structure and can have a first inner diameter. The funnel mouth can be perpendicular to the longitudinal axis and can have a second inner diameter. The first inner diameter of the elongate body can be smaller than the second inner diameter of the funnel mouth. As such, the second portion of the obstruction, which can be received through the funnel mouth, can be larger than the first portion of the obstruction, which can be received through the elongate body.

The guide wire can be configured such that there is a certain distance between the distal end of the guide wire and the funnel mouth of the aspiration catheter such that the guide wire does not extend past the distal end of the catheter.

Each of the auger fins of the guide wire can be the same size. The auger fins can be sized to have minimal clearance with the first inner diameter of the elongate body in order to displace the obstruction in the proximal direction jointly with aspiration force. The one or more fragments of the obstruction can be aspirated through the funnel mouth and in the proximal direction around the auger fins.

Another example system configured to retrieve an obstruction from a blood vessel can include an aspiration catheter and an injection catheter. The aspiration catheter can include an elongate body including a proximal end, a distal end, and a lumen sized to receive at least a first portion of the obstruction therein. The aspiration catheter can also include a funnel continuous with the distal end of the elongate body and including a funnel mouth sized to receive at least a second portion of the obstruction therein. The injection catheter can be configured in the lumen of the elongate body, and can include an orifice positioned approximate the distal end of the injection catheter. The injection catheter can be configured to produce a jet through the orifice such that the jet macerates the obstruction into fragments as one or more of the fragments of the obstruction are aspirated in the proximal direction around the injection catheter and through the aspiration catheter.

The elongate body of the aspiration catheter can have a single continuous structure and can have a first inner diameter. The funnel mouth can be perpendicular to the longitudinal axis and can have a second inner diameter. The first inner diameter of the elongate body can be smaller than the second inner diameter of the funnel mouth. As such, the second portion of the obstruction, which can be received through the funnel mouth, can be larger than the first portion of the obstruction, which can be received through the elongate body.

The distal end of the injection catheter can be closed. The injection catheter can be configured such that there is a certain distance between the distal end of the injection catheter and the funnel mouth of the aspiration catheter such that the injection catheter does not extend past the distal end of the catheter. The injection catheter can be either integral with the elongate body, or separate from the elongate body and sized to slide through the lumen of the elongate body.

An example method of retrieving an obstruction from a blood vessel can include one or more of the following steps executed by a person skilled in the pertinent art. The example method can include positioning an aspiration catheter such that a funnel mouth of a funnel disposed at the distal end of the aspiration catheter is proximate the obstruction. A negative pressure region can be created within the blood vessel by aspirating through a lumen of the aspiration catheter until a proximal portion of the obstruction becomes lodged in the funnel mouth. A clot macerating device, such as a guide wire or injection catheter, can be positioned inside the lumen of the aspiration catheter, and can be actuated thereby macerating the obstruction into fragments while simultaneously aspirating one or more of the fragments of the obstruction in the proximal direction through the aspiration catheter.

The clot macerating device can be configured such that there is a certain distance between the distal end of the clot macerating device and the funnel mouth of the aspiration catheter, and the one or more fragments of the obstruction can be aspirated in the proximal direction around the clot macerating device.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A is an illustration of an example system during an example clot aspiration step according to aspects of the present invention.

FIG. 1B is an illustration of an example system during an example clot aspiration step according to aspects of the present invention.

FIG. 1C is an illustration of an example system during an example clot aspiration step according to aspects of the present invention.

FIGS. 2A-2E are illustrations of example treatment steps including the example system and example step in FIG. 1A-1C according to aspects of the present invention.

FIG. 3 is an illustration of another example system during another example clot aspiration step according to aspects of the present invention.

FIG. 4 is a cross-sectional view of the example system in FIG. 3 according to aspects of the present invention.

FIG. 5 is an illustration of another example system during another example clot aspiration step according to aspects of the present invention.

FIG. 6 is a cross-sectional view of the example system in FIG. 5 according to aspects of the present invention.

FIGS. 7A-7C are illustrations of example treatment steps including the example system and example step in FIG. 3 according to aspects of the present invention.

FIG. 8A is a diagram of an example method of treatment including the example system and example steps in FIGS. 1A and 2A-2E.

FIG. 8B is a diagram of an example method of treatment including either the example system and example step in FIG. 1B or FIG. 1C.

FIG. 9 is a diagram of an example method of treatment including either the example system and example steps in FIGS. 3, 4, and 7A-7C, or FIGS. 5-6.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

The example systems and methods of treatment described herein generally involve delivering an aspiration catheter and a clot macerating device, such as an auger-style or wire loop guide wire or an injection catheter, to a blood vessel obstruction, e.g., a clot. The clot can be aspirated, by the aspiration catheter, in a proximal direction through a lumen of the aspiration catheter and around either the guide wire or the injection catheter. The example systems and methods can fully ingest a clot at a treatment site and aspirate the clot through the catheter while ensuring the clot, or smaller fragments of the clot, do not break off or become uncorked from the catheter tip.

In some such examples including a guide wire, the guide wire may have one or more auger fins disposed at the distal end of the guide wire. The guide wire may be torqued such that the clot can be macerated, and macerated fragments of the clot can be conveyed in the proximal direction around the auger fins and through the aspiration catheter. Simultaneous aspiration can also be conducted through the aspiration catheter to further help aspirate the clot fragments in the proximal direction through the aspiration catheter.

In other such examples including a guide wire, the guide wire may have one or more wire loops disposed at the distal end of the guide wire. The guide wire may be torqued such that the clot can be macerated, and macerated fragments of the clot can be conveyed via simultaneous aspiration in the proximal direction around and/or through the wire loop(s) and through the aspiration catheter.

In other such examples including an injection catheter, the injection catheter may have an orifice at or near the distal end of the injection catheter such that a solution (e.g., saline) may be injected through the orifice to create a jet. The jet can help to macerate the clot, while simultaneous aspiration can help to aspirate the macerated clot fragments in the proximal direction through the aspiration catheter.

Various example systems and methods are presented herein. Features from each example are combinable with other examples as understood by persons skilled in the pertinent art.

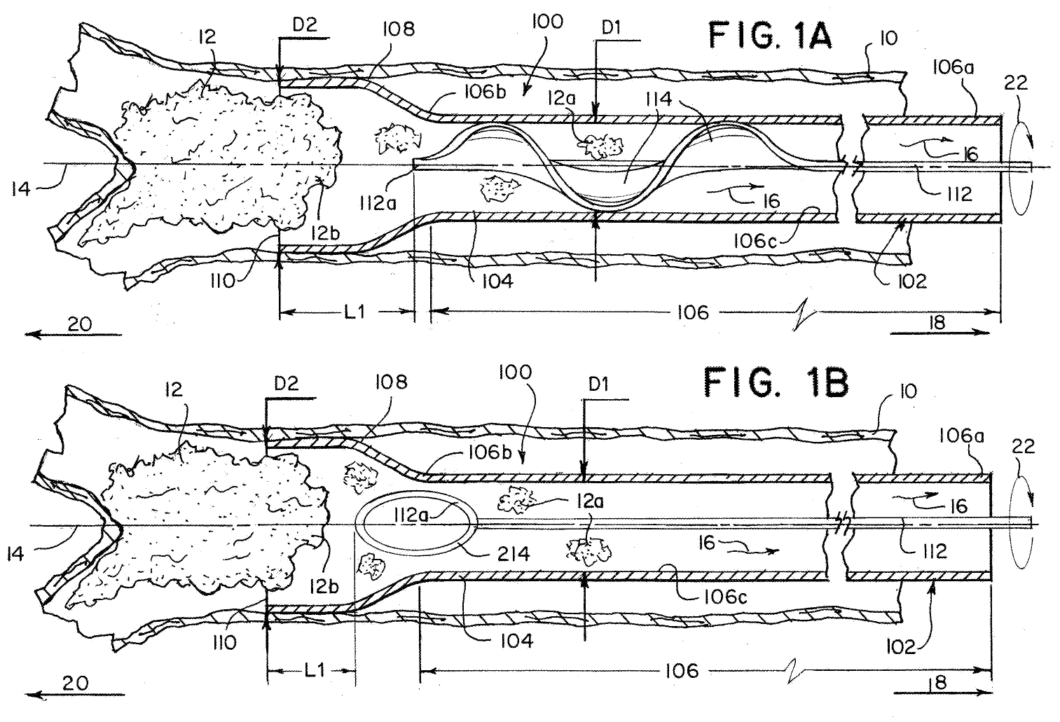

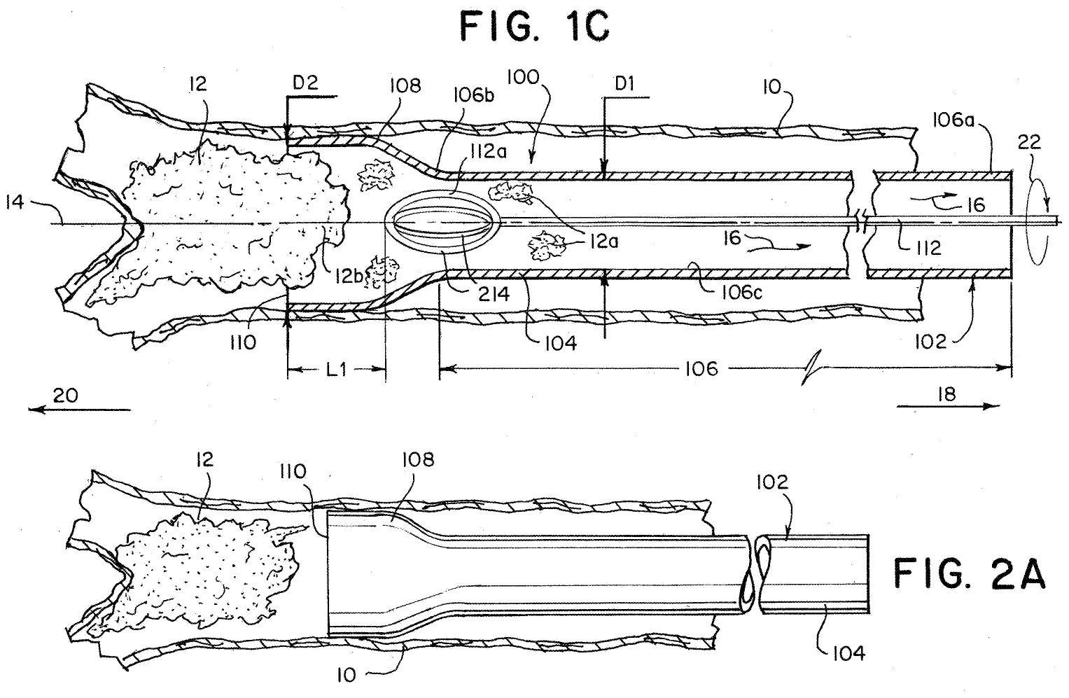

FIG. 1A is an illustration of an example system 100 during an example clot aspiration step. The system 100 includes a device 102 configured to retrieve a clot 12 from a blood vessel 10. The device 102 includes an aspiration catheter 104 and a guide wire 112. The aspiration catheter 104 includes an elongate body 106 and a funnel 108. The elongate body 106 includes a proximal end 106a, a distal end 106b, and a lumen 106c sized to receive at least a first portion 12a of the clot 12. The funnel 108 is continuous with the distal end 106b of the elongate body 106 and includes a funnel mouth 110 sized to receive at least a second portion 12b (e.g., a proximal portion) of the clot 12 therein. The guide wire 112 is sized to slide through the lumen 106c of the elongate body 106 and includes one or more auger fins 114 disposed at a distal end 112a of the guide wire 112.

During the illustrated step, negative pressure from aspiration 16 creates a force on the clot 12 to draw the proximal portion 12b of the clot 12 into the funnel mouth 110 of the funnel 108. Simultaneously, guide wire 112 is torqued 22, such that the auger fins 114 can macerate the clot 12 into fragments. The auger fins 114 of guide wire 112 can convey the macerated fragments of clot 12 in a proximal direction 18 through the aspiration catheter 104. Simultaneous aspiration 16 can be used along with the torquing of the guide wire 112 to help support or supplement such conveyance of the macerated fragments.

As shown in FIG. 1A, the elongate body 106 includes a single continuous structure having an inner diameter D1. The distal end 106b of elongate body 106 includes a continuous or seamless connection with the funnel 108. The funnel mouth 110 of the funnel 108 can be perpendicular to the longitudinal axis 14 and has an inner diameter D2. As illustrated, inner diameter D2 of funnel mouth 110 is larger than inner diameter D1 of elongate body 106 such that funnel mouth 110 can receive larger portions of clot 12, e.g., proximal portion 12b, than can elongate body 106, e.g., first portion 12a. That is, once aspiration 16 has drawn clot 12 into the funnel mouth 110, guide wire 112 can be torqued such that the auger fins 114 can macerate the clot 12 and convey smaller fragments of the clot in the proximal direction 18 through the aspiration catheter 104.

The guide wire 112 is positioned in the lumen 106c of elongate body 106 such that there is a distance L1 between the distal end 112a of the guide wire 112 and the funnel mouth 110 of the aspiration catheter 104. This distance L1 helps to ensure the distal end 112a of the guide wire 112 does not inadvertently pierce or puncture the clot 12 such that a portion of the clot 12 might break off from the clot 12 outside of, or distal to, the funnel mouth 110. Such broken portion of the clot 12 could re-block the blood vessel 10 and provide a challenge for later retrieval. Instead, the distance L1 helps to ensure the clot 12 may be aspirated 16 in a proximal direction 18 such that it becomes lodged or corked in the funnel mouth 110 before the auger fins 114 start to macerate the clot 12. Additionally, this process helps to ensure the clot 12 is macerated inside the funnel 108.

The auger fins 114 of guide wire 112 can each be of the same size, and can be sized such that there is minimal clearance with the inner diameter D1 of elongate body 106. Providing such minimal clearance helps to ensure the auger fins 114 can independently convey clot fragments in the proximal direction 18 of the aspiration catheter 104, but can also do so jointly with aspiration force 16.

FIG. 1B is an illustration of another example system 100 during an example clot aspiration step. Many of the features of system 100 as shown in FIG. 1B may be the same as or similar to those as shown in FIG. 1A and described above. In some embodiments, however, rather than guide wire 112 include one or more auger fins 114 disposed at its distal end 112a, guide wire 112 may instead have a wire loop 214 disposed at its distal end 112a. The wire loop 214 may be configured such that when guide wire 112 is torqued, the wire loop 214 macerates the clot 12 into clot fragments. That is, once aspiration 16 has drawn clot 12 into the funnel mouth 110, guide wire 112 can be torqued such that the wire loop 214 can macerate the clot 12 while simultaneous aspiration 16 can convey smaller fragments of the clot in the proximal direction 18 around and/or through the wire loop 214 and through the aspiration catheter 104.

FIG. 1C is an illustration of another example system 100 during an example clot aspiration step. Many of the features of system 100 as shown in FIG. 1C may be the same as or similar to those as shown in FIGS. 1A and 1B and described above. In some embodiments, however, guide wire 112 may include a series of wire loops 214 at its distal end 214. Depending on the configuration (e.g., the size) of the clot 12, the series of wire loops 214 may provide additional structure to macerate the clot 12 into fragments while simultaneous aspiration 16 may convey the smaller clot fragments in the proximal direction 18 around and/or through the series of wire loops 214 and through the aspiration catheter 104.

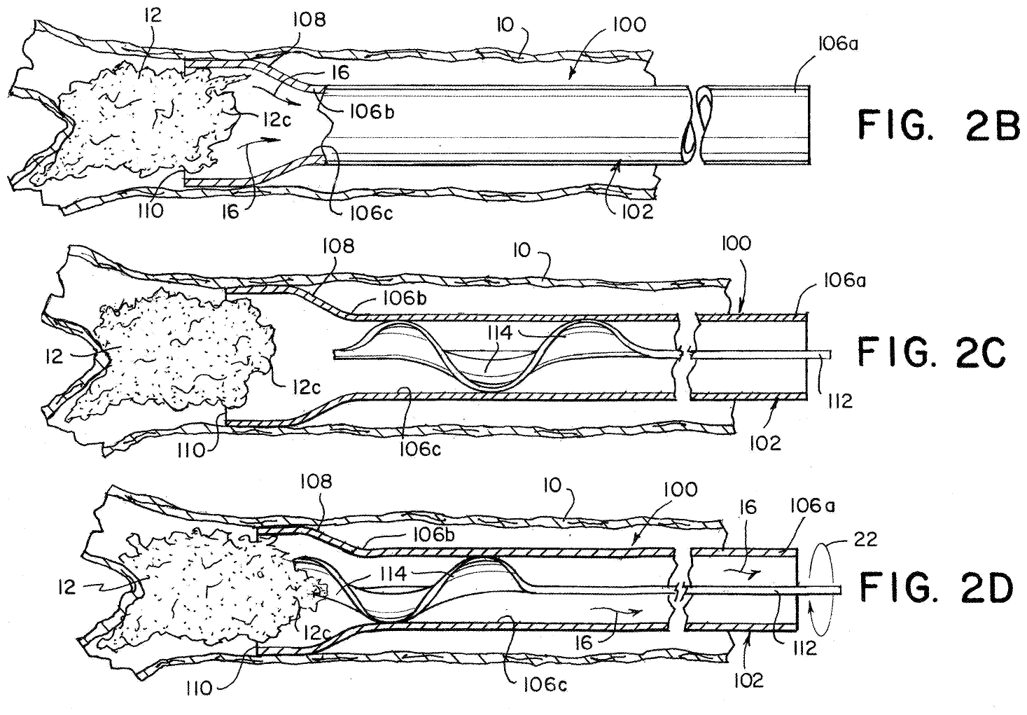

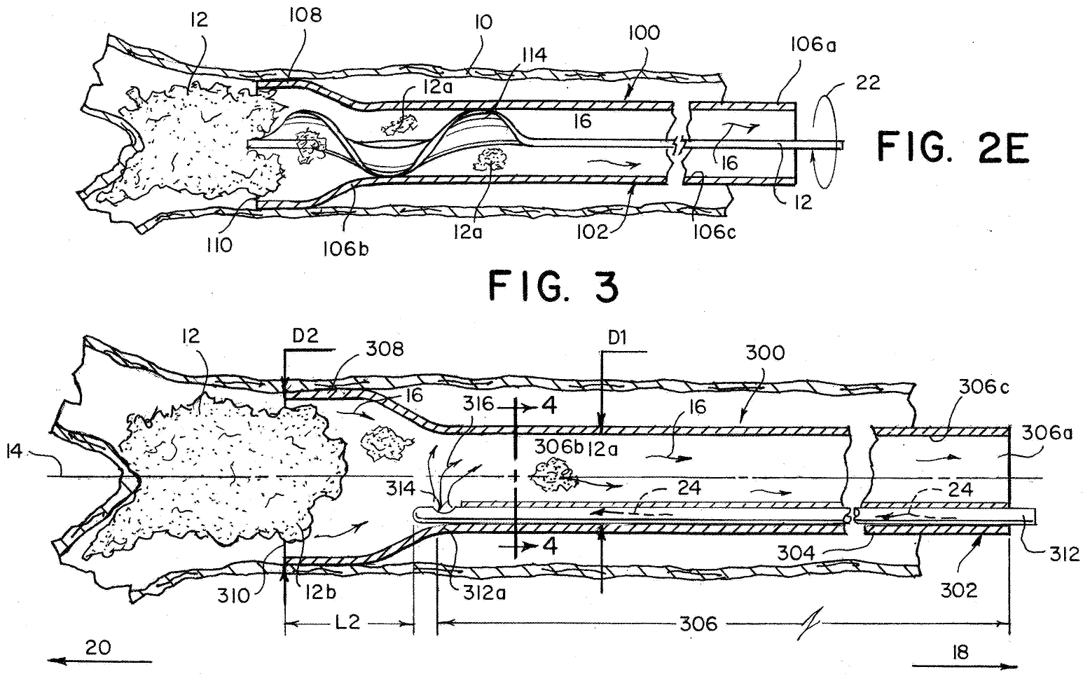

FIGS. 2A through 2E are illustrations of example treatment steps including the example system and example step illustrated in FIG. 1A-1C.

FIG. 2A illustrates the funnel mouth 110 of the aspiration catheter 104 positioned near, and in a proximal direction 18 from, a clot 12 at a bifurcation of a blood vessel 10.

FIG. 2B illustrates an aspiration force 16 applied in a proximal direction 18 through the aspiration catheter 104 until a proximal portion 12b of the clot 12 becomes lodged or corked in the funnel mouth 110 of the aspiration catheter 104.

FIG. 2C illustrates a guide wire 112 being positioned inside a lumen 106c of an elongate body 106 of aspiration catheter 104, and near, and in a proximal direction 18 from, the funnel mouth 110 of the aspiration catheter 104. The guide wire 112, which includes auger fins 114 at a distal end 112a thereof, is positioned such that there is a distance L1 (FIG. 1A) between the distal end 112a of the guide wire 112 and the funnel mouth 110.

FIG. 2D illustrates applying torque 22 to the guide wire 112 such that the auger fins 114 begin to spin into and/or against the proximal portion 12b of clot 12. Simultaneously, aspiration force 16 is again applied in a proximal direction 18 through the aspiration catheter 104 to help aspirate the macerated clot in the proximal direction 18 as clot fragments start breaking apart from clot 12.

FIG. 2E illustrates clot fragments, or first portions 12a, of clot 12 being macerated by the torqued auger fins 114 while simultaneously aspirated in a proximal direction 18 through aspiration catheter 104. Throughout the above-described method of treatment, clot 12 can remain lodged or corked in funnel mouth 110 of the aspiration catheter 104.

FIG. 3 is an illustration of another example system 300 during an example clot aspiration step. The system 300 includes a device 302 configured to retrieve a clot 12 from a blood vessel 10. The device 302 includes aspiration catheter 304 and an injection catheter 312. The aspiration catheter 304 includes an elongate body 306 and a funnel 308. The elongate body 306 includes a proximal end 306a, a distal end 306b, and a lumen 306c sized to receive at least a first portion 12a of the clot 12. The funnel 308 is continuous with the distal end 306b of the elongate body 306 and includes a funnel mouth 310 sized to receive at least a second portion 12b (e.g., a proximal portion) of the clot 12 therein. The injection catheter 312 is configured in the lumen 306c of the elongate body 306, and includes an orifice 314 positioned approximate a distal end 312a of the injection catheter 312.

During the illustrated step, negative pressure from aspiration 16 creates a force on the clot 12 to draw the proximal portion 12b of the clot 12 into the funnel mouth 310 of the funnel 308. Simultaneously, a solution (e.g., saline) is injected through the orifice 314 to produce a jet 316 that macerates the clot 12 into fragments as one or more of the fragments of the clot 12 are aspirated in a proximal direction 18 through the aspiration catheter 304.

As shown in FIG. 3, the distal end 312a of the injection catheter 312 is closed other than the orifice 314 positioned approximate the distal end 312a. The injection catheter 312 is configured in the lumen 106c of the elongate body 106 such that there is a distance L2 between the distal end 312a of the injection catheter 312 and the funnel mouth 110 of the aspiration catheter 104. This distance L2 helps to ensure the jet 316 does not inadvertently break off a portion of the clot 12 outside of, or distal to, the funnel mouth 310. Instead, the distance L2 helps to ensure clot 12 may be aspirated 16 in a proximal direction 18 such that it becomes lodged or corked in the funnel mouth 310 before the jet 316 starts to macerate the clot 12. Additionally, this process helps to ensure the clot 12 is macerated inside the funnel 308.

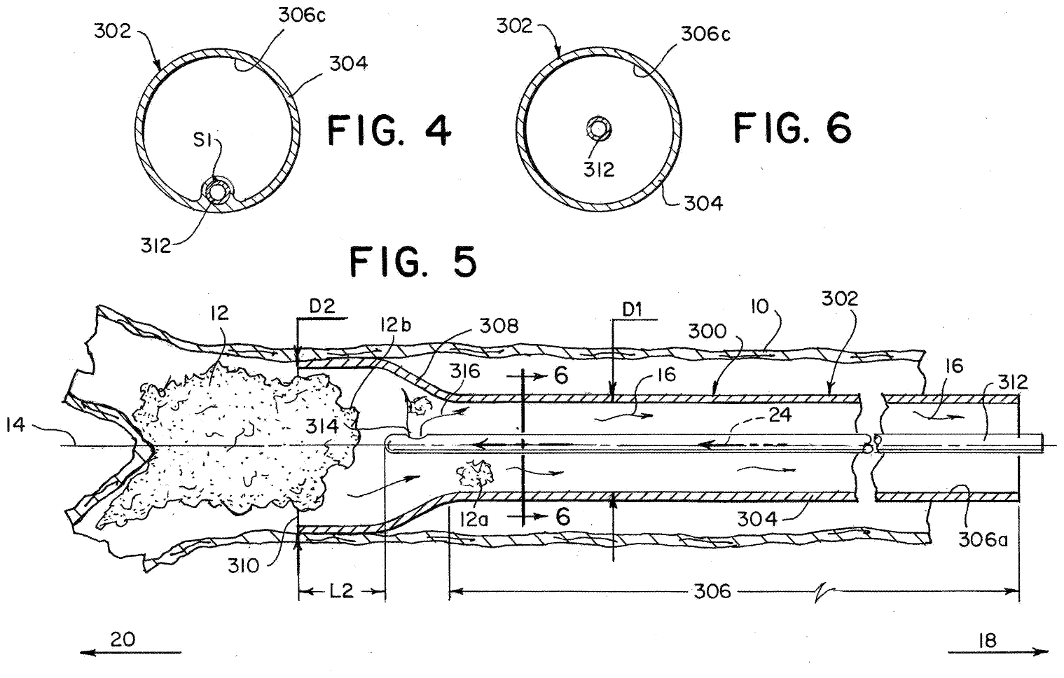

The injection catheter 312 can be configured to be either integral with the elongate body 306, as illustrated in FIGS. 3 and 4, or separate from the elongate body 306 and sized to slide through the lumen 306c, as further described below with respect to FIGS. 5 and 6.

As shown in FIG. 3, the injection catheter 312 is integral with the elongate body 306 such that it is connected to an inner wall of the elongate body 306. As shown in FIG. 4, illustrating a cross-sectional view of the device 302, the injection catheter 312 can be connected to or integrated with an inner wall of the elongate body 306, for example, by a sleeve 51, such that aspiration catheter 304 and injection catheter 312 can be configured as a single device for simultaneous delivery to a treatment site of a patient.

Alternatively, as shown in FIGS. 5 and 6, the injection catheter 312 can be separate from the aspiration catheter 304. That is, as shown in FIGS. 5 and 6, injection catheter 312 can be positioned within the lumen 306c of the elongate body 306, for example, to modify the placement of the jet 316 in relation to the funnel mouth 310, the funnel 308, the clot 12, etc.

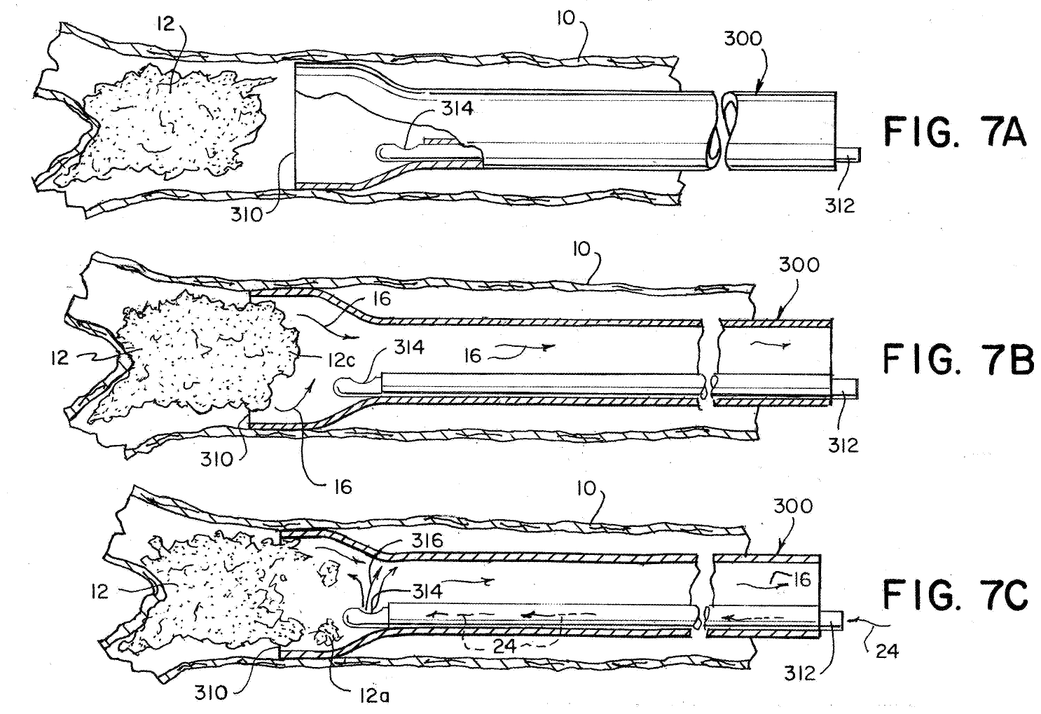

FIGS. 7A through 7C are illustrations of example treatment steps including the example system and example step illustrated in FIG. 3.

FIG. 7A illustrates the funnel mouth 310 of the aspiration catheter 304 positioned near, and in a proximal direction 18 from, a clot 12 at a bifurcation of a blood vessel 10. The injection catheter 312 is integral with the aspiration catheter 304.

FIG. 7B illustrates an aspiration force 16 applied in a proximal direction 18 through the aspiration catheter 304 until a proximal portion 12b of the clot 12 becomes lodged or corked in the funnel mouth 310 of the aspiration catheter 304.

FIG. 7C illustrates injecting a solution through the injection catheter 312 and through orifice 314 of injection catheter 312 thereby producing a jet 316 to start macerating the proximal portion 12b of the clot 12, while the macerated fragments, or first portions 12a, of the clot 12 are aspirated in the proximal direction 18 through the aspiration catheter 304.

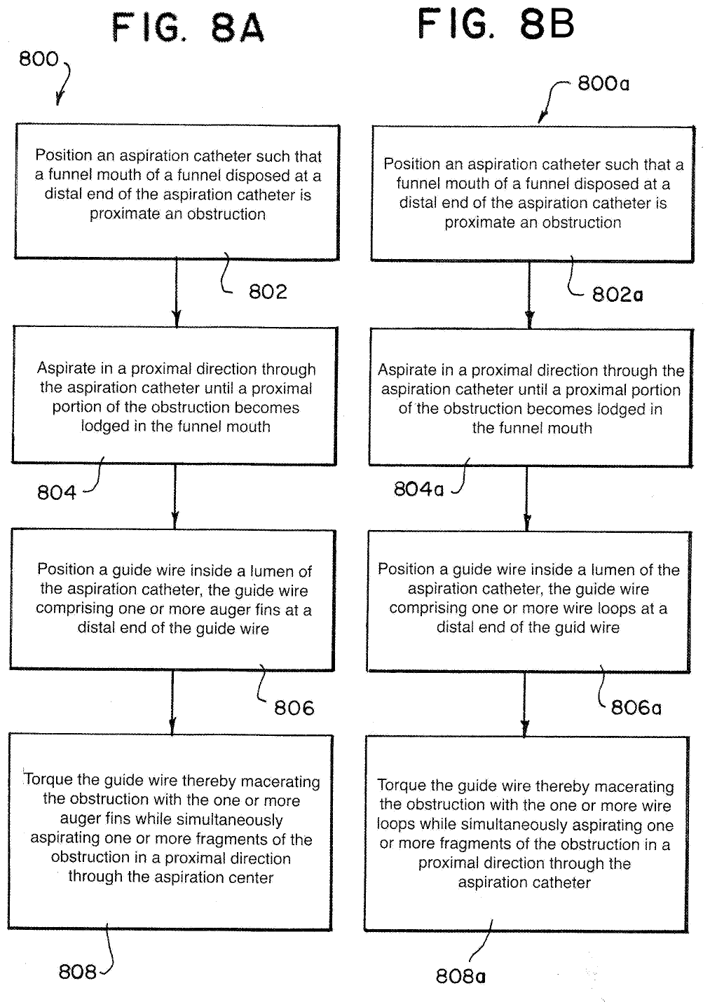

FIG. 8A is a diagram of an example method of treatment 800 including the example system and example steps illustrated in FIGS. 1A and 2A-2E.

In block 802, an operator (e.g., a physician) can position the aspiration catheter 104 in a blood vessel 10 of a patient such that the funnel mouth 110 of the funnel 108 is proximate the clot 12 (FIG. 2A).

In block 804, the operator can then begin to aspirate 16 in a proximal direction 18 through the aspiration catheter 104 until a proximal portion 12b of the clot 12 becomes lodged in the funnel mouth 110 (FIG. 2B).

In block 806, the operator can position the guide wire 112 inside a lumen 106c of the aspiration catheter 104. The guide wire 112 can include one or more auger fins 114 disposed at a distal end 112a of the guide wire 112 (FIG. 2C).

In block 808, the operator can torque the guide wire 112 thereby spinning or screwing the auger fins 114 such that they begin to macerate the clot 12 (FIG. 2D). Simultaneously, the operator again aspirates 16 in the proximal direction 18 to aspirate the macerated clot fragments of the clot 12 in the proximal direction 18 around the auger fins 114 and through the aspiration catheter 104 (FIG. 2E).

FIG. 8B is a diagram of an example method of treatment 800a including the example system and example step illustrated in FIG. 1B or FIG. 1C. The descriptions of blocks 802a and 804a of method 800a may be the same as or similar to the respective descriptions of blocks 802 and 804 of method 800, and as such, are not repeated herein for brevity.

In block 806a, the operator can position the guide wire 112 inside a lumen 106c of the aspiration catheter 104. The guide wire 112 can include one or more wire loops 214 disposed at a distal end 112a of the guide wire 112.

In block 808a, the operator can torque the guide wire 112 thereby spinning the one or more wire loops 214 such that the wire loop(s) 214 begin to macerate the clot 12. Simultaneously, the operator may aspirate 16 in the proximal direction 18 around and/or through the wire loop(s) 214 and through the aspiration catheter 104.

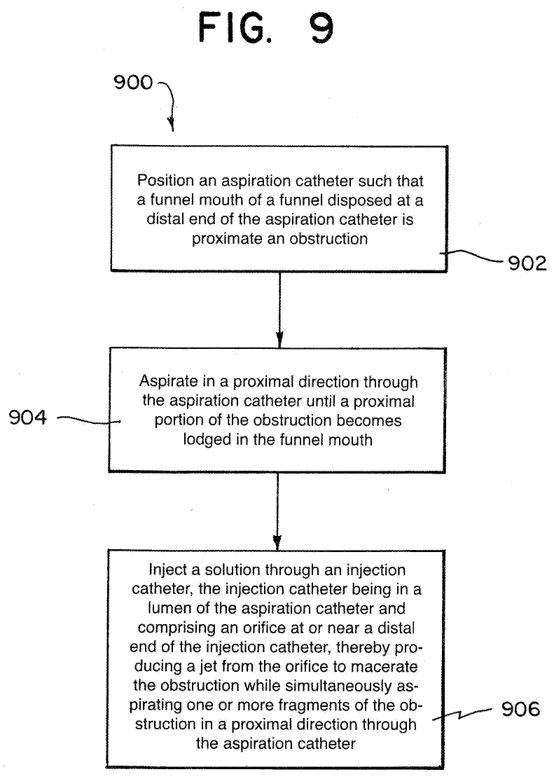

FIG. 9 is a diagram of an example method of treatment 900 including the example system and example steps illustrated in either FIGS. 3, 4, and 7A-7C, or FIGS. 5-6.

In block 902, an operator (e.g., a physician) can position the aspiration catheter 304 in a blood vessel 10 of a patient such that the funnel mouth 310 of the funnel 308 is proximate the clot 12 (FIG. 7A). As described above, the aspiration catheter 304 can include an integrated injection catheter 312 (FIGS. 3, 4, 7A-7C), or an injection catheter 312 that is separate from the aspiration catheter 304 (FIGS. 5-6). If using an aspiration catheter 304 with an integrated injection catheter 312, the operator can deliver the single device 302 to the treatment site of the patient. Alternatively, if using an aspiration catheter 304 with a separate injection catheter 312, the operator may need to separately deliver the aspiration catheter 304 and the injection catheter 312 to the treatment site. For example, the operator can first deliver the aspiration catheter 304 to the treatment site and then slide the injection catheter 312 into the aspiration catheter 304.

In block 904, the operator can begin to aspirate 16 in a proximal direction 18 through the aspiration catheter 304 until a proximal portion 12b of the clot 12 becomes lodged in the funnel mouth 310 (FIG. 7B).

In block 906, the operator can inject a solution through the injection catheter 312 thereby producing a jet 316 through the orifice 314 of the injection catheter 312. The jet 316 can macerate the clot 12 while the operator simultaneously aspirates the macerated clot fragments of the clot 12 in a proximal direction 18 through the aspiration catheter 304 (FIG. 7C).

The descriptions contained herein are examples of embodiments of the invention and are not intended in any way to limit the scope of the invention. As described herein, the invention contemplates many variations and modifications of structures and methods, including alternative materials, alternative configurations of component parts, and alternative method steps. Modifications and variations apparent to those having skill in the pertinent art according to the teachings of this disclosure are intended to be within the scope of the claims which follow.

What is claimed is:

1. A device configured to retrieve an obstruction from a blood vessel, the device comprising:
an aspiration catheter comprising:
an elongate body comprising a proximal end, a distal end, and a lumen sized to receive at least a first portion of the obstruction therein; and
a funnel continuous with the distal end of the elongate body and comprising a funnel mouth sized to receive at least a second portion of the obstruction therein; and
a guide wire sized to slide through the lumen and comprising one or more auger fins disposed at a distal end of the guide wire;
wherein the guide wire is configured to be torqued such that the one or more auger fins macerate the obstruction into fragments as one or more of the fragments of the obstruction are aspirated in a proximal direction through the aspiration catheter.

2. The device of claim 1, wherein the elongate body comprises a single continuous structure.

3. The device of claim 1, wherein the funnel mouth is perpendicular to the longitudinal axis.

4. The device of claim 1, wherein the second portion of the obstruction is larger than the first portion of the obstruction.

5. The device of claim 1, wherein the elongate body comprises a first inner diameter and the funnel mouth comprises a second inner diameter, the first inner diameter being smaller than the second inner diameter.

6. The device of claim 5, wherein each of the one or more auger fins are sized to have a minimal clearance with the first inner diameter in order to displace the obstruction in the proximal direction jointly with aspiration force.

7. The device of claim 1, wherein each of the one or more auger fins are the same size.

8. The device of claim 1, wherein the one or more fragments of the obstruction are configured to be aspirated in the proximal direction around the one or more auger fins.

9. The device of claim 1, wherein the one or more fragments of the obstruction are configured to be aspirated through the funnel mouth of the aspiration catheter.

10. The device of claim 1, wherein the guide wire is configured such that there is a distance between the distal end of the guide wire and the funnel mouth of the aspiration catheter.

11. A device configured to retrieve an obstruction from a blood vessel, the device comprising:
an aspiration catheter comprising:
an elongate body comprising a proximal end, a distal end, and a lumen sized to receive at least a first portion of the obstruction therein; and
a funnel continuous with the distal end of the elongate body and comprising a funnel mouth sized to receive at least a second portion of the obstruction therein; and
an injection catheter in the lumen and comprising an orifice positioned approximate a distal end of the injection catheter;
wherein the injection catheter is configured to produce a jet through the orifice such that the jet macerates the obstruction into fragments as one or more of the fragments of the obstruction are aspirated in a proximal direction through the aspiration catheter.

12. The device of claim 11, wherein the elongate body comprises a single continuous structure.

13. The device of claim 11, wherein the funnel mouth is perpendicular to the longitudinal axis.

14. The device of claim 11, wherein the elongate body comprises a first inner diameter and the funnel mouth comprises a second inner diameter, the first inner diameter being smaller than the second inner diameter.

15. The device of claim 11, wherein the one or more fragments of the obstruction are configured to be aspirated in the proximal direction around the injection catheter.

16. The device of claim 11, wherein the distal end of the injection catheter is closed.

17. The device of claim 11, wherein the injection catheter is configured such that there is a distance between the distal end of the injection catheter and the funnel mouth of the aspiration catheter.

18. The device of claim 11, wherein the injection catheter is configured to be either:
integral with the elongate body; or
separate from the elongate body and sized to slide through the lumen.

19. A method of retrieving an obstruction from a blood vessel, the method comprising:
positioning an aspiration catheter such that a funnel mouth of a funnel disposed at a distal end of the aspiration catheter is proximate the obstruction;
wherein the aspiration catheter comprises an elongate body; and
wherein the funnel is continuous with a distal end of the elongate body;
aspirating in a proximal direction through the aspiration catheter until a proximal portion of the obstruction becomes lodged in the funnel mouth;
positioning a clot macerating device inside a lumen of the aspiration catheter; and
actuating the clot macerating device thereby macerating the obstruction into fragments while simultaneously aspirating one or more of the fragments of the obstruction in a proximal direction through the aspiration catheter.

20. The method of claim 19, wherein the clot macerating device is configured such that there is a distance between the distal end of the clot macerating device and the funnel mouth of the aspiration catheter, and wherein the one or more fragments of the obstruction are configured to be aspirated in the proximal direction around the clot macerating device.
